(19) United States
(12) Patent Application Publication
(10) Pub. No.: US 2009/0041442 A1
(43) Pub. Date: Feb. 12, 2009

(54) Thermal Vaporizer Apparatus

(76) Inventor: Martin D. Rouse, JR., Escondido, CA (US)

Correspondence Address: GORDON & REES LLP, 101 WEST BROADWAY, SUITE 1600, SAN DIEGO, CA, 92101, US

(21) Appl. No.: 12/224,931

(22) PCT Filed: Mar. 6, 2007
(86) PCT No.: PCT/US07/05590

§ 371 Date: Oct. 7, 2008

Related U.S. Application Data
(60) Provisional application No. 60/779,826, filed on Mar. 6, 2006.

(51) Int. Cl. A61L 9/00

(52) U.S. Cl. 392/386

(57) ABSTRACT

The present invention relates to a smokeless thermal vaporizer apparatus for extracting and vaporizing essential ingredients from natural herbal medicines for inhalation to prevent and alleviate pains and diseases. The thermal vaporizer apparatus includes a heating device and a handheld delivery device. The heating device has a heating element disposed within a thermally insulated housing with an access opening on front and a switch with a variable voltage controller on the front surface of the housing to regulating the amount heat generated by the heating element. The heating element is securely mounted to the housing with the rear end attached to the housing and the front end positioned in the center of the access opening. The handheld delivery device includes a vaporization chamber for holding herbal medicine and receiving smokeless hot air from the heating element of the heating device and a mouthpiece for inhalation. The vaporization chamber and the mouthpiece are linked together with a vapor delivery hose.

### TECHNICAL FIELD

The present invention relates to a thermal vaporizer apparatus for extracting and vaporizing essential ingredients from natural herbal medicines for inhalation to prevent and alleviate pains and diseases.

### BACKGROUND OF THE INVENTION

Herbal medicine has been widely used for the treatment of medical conditions and diseases in the world for thousands of years. During the last two decades, there has been seen a worldwide upsurge in the use of traditional medicine and complementary and alternative medicine in both developed and developing countries. In Africa, nearly 80% of the population in rural areas depends on traditional medicine to meet their primary health care needs. In India, the corresponding figure is about 65%. In developed countries, including the United States, nearly one in every two people has used herbal medicines at least once in the last ten years. In China, traditional herbal medicine accounts for 30%-50% of the total medicinal consumption.

Herbal medicines are commonly administered through oral delivery using the extracts containing the essential ingredients from herbal medicines or through pulmonary delivery if the therapeutically active components are volatile enough. Compared with oral delivery route, pulmonary delivery offers several distinct advantages, among them the circumvention of first pass metabolism in the gastrointestinal tract and access to a high concentration of narrow blood vessels with large surface area available for transport. This large surface area provides rapid systemic absorption when compared with the oral route of administration, thus resulting in rapid onset of action at substantially lower doses.

The conventional technique for extracting and vaporizing the essential ingredients in herbal plants for pulmonary delivery has been combustion such as smoking. This combustion method produces a wide spectrum of toxic and carcinogenic by-products, which are unavoidably inhaled along with the beneficial ingredients and introduce significant health risks to the patients especially after prolonged use. Additionally, the combustion method is also not economical.

The high and uncontrolled combustion temperature causes the unnecessary destruction of the majority of the essential ingredients, often rendering them useless.

Recently, vaporization technologies have been successfully applied to overcome these shortcomings by heating the herbal materials directly or indirectly to a temperature just high enough to get the therapeutically active ingredients vaporized, but without reaching the point of combustion. Among them, one common technology is convective vaporization in which a clean hot air passes through the packed herbal materials in a vaporization chamber to provide the thermal energy required for vaporization of the essential ingredients. The herbal materials are indirectly heated by a hot air whose temperature can be precisely controlled to avoid reaching the combustion point.

Several types of thermal vaporizer apparatuses which use this convective vaporization technology have been developed and some of them are commercially available. While the structural arrangements of these vaporization apparatuses, at first appearance, may have similarities with the present invention, they differ in material respects and all have some limitations. Accordingly, an object of the present invention is to provide a thermal vaporizer apparatus to overcome these limitations, which is economical to build and convenient to operation.

### SUMMARY OF THE INVENTION

In accordance with the present invention, a thermal vaporizer apparatus is provided for the extraction and vaporization of the volatile essential ingredients from natural herbal plants for inhalation using convective vaporization technology. In use of the apparatus of the present invention, a smokeless hot air is passed through the herbal plants in a vaporization chamber at a temperature just high enough to get the therapeutically active ingredients vaporized but without reaching the point of combustion.

In one embodiment, the thermal vaporizer apparatus comprises at least one heating device which generates smokeless hot air for use in the extraction and vaporization of the essential ingredients in herbal medicines. The heating device includes an elongated heating element, a switch with a voltage controller (such as a potentiometer), and a housing with a heating chamber with a front access opening. The elongated heating element is securely mounted within the heating chamber by inserting its rear end into the center hole on the rear wall of the heating chamber, with the front end pointed towards the center of the front access opening. To maximize heating efficiency, the heating element is mounted with an upward angle of from about 1 to about 29° from horizontal. The rotary switch is attached to the front wall of the housing, functioning as both an on/off switch and an adjustable potentiometer for regulating the amount of heat generation by the heat element, thus the temperature of the hot air.

In another embodiment, the thermal vaporizer apparatus further comprises a handhold vapor delivery device which includes a vaporization chamber, with an inlet and an outlet, for holding herbal plants and receiving smokeless hot air from the heating element of the heating, an inhalation mouthpiece, and a flexible vapor delivery hose connected to the outlet of the vaporizer chamber on one end and to the inhalation mouthpiece on the other end. The vaporization chamber also includes a barrier within the cylindrical bore of the vaporization chamber and close to the outlet. The barrier is used to minimize the passage of the particles, including the herbal plants and the particles generated from these herbal plants, in the vaporization chamber into the vapor delivery hose. In one aspect, the barrier is a section of the cylindrical body of the vaporization chamber, having an inner dimension sufficiently reduced to prevent the passage of the particles. In another aspect, the barrier is a dimpled section of the cylindrical body of the vaporization chamber.

In another embodiment, the vaporization chamber of the handhold vapor delivery device further comprises a second barrier to prevent the inadvertent spillage of the packed herbal plants from the vaporization chamber

### BRIEF DESCRIPTION OF THE DRAWINGS

FIG. 1 is a perspective view of a heating device of a thermal vaporizer apparatus.

FIG. 2 is a sectional representation of a heating device of a thermal vaporizer apparatus.

FIG. 3 is a sectional view of a heating element.

FIG. 4 is a sectional view of a retainer for attachment of the heating element to the housing in the heating device.

FIG. 5 is a perspective view of a handhold delivery device of a thermal vaporizer apparatus.

FIG. 6 is a sectional representation of the vaporization chamber of the handhold delivery device of a thermal vaporizer apparatus.

FIG. 7 is a sectional representation of the vaporization chamber of the handhold delivery device of a thermal vaporizer apparatus.

FIG. 8 is a sectional representation of the vaporization chamber of the handhold delivery device of a thermal vaporizer apparatus.

FIG. 9 is a sectional representation of a heating device with an height adjustment control which can be used to adjust the angle of the heating element relative to the horizontal surface.

### DETAILED DESCRIPTION OF THE INVENTION

The present invention relates to a thermal vaporizer apparatus 1 for extracting and vaporizing the volatile essential ingredients from herbal plants for pulmonary administration to treat medical conditions and diseases. The thermal vaporizer apparatus 1 includes a heating device 2 and a handhold delivery device 3. The electrically powered heating device 2 provides smokeless hot air at a temperature just high enough to .get the therapeutically active ingredients vaporized from herbal plants, thus potentially providing maximum benefits to the patient without deleterious side effects commonly associated with smoking.

### Heating Device

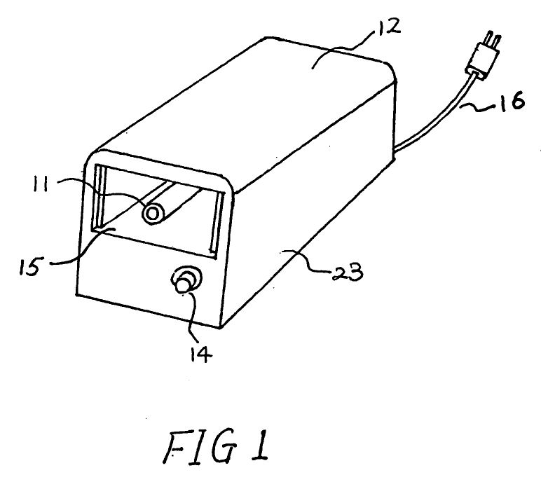

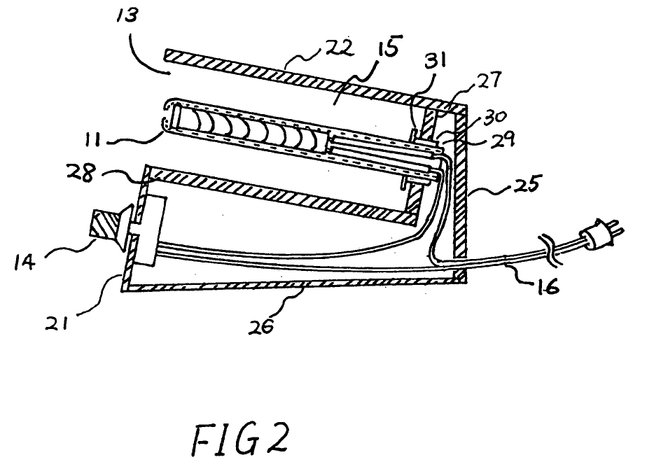

As illustrated in FIGS. 1 and 2, the heating device 2 contains a housing 12 with a electric circuit 16, an elongated heating element 11 for providing smokeless hot air for extracting and vaporizing the active ingredients from the herbal plants for inhalation, and a switch 14 with a voltage controller for regulating the amount of heat generated by the heating element 11, thus the temperature of the hot air. The housing 12 is formed by a polarity of housing walls, including a front 21 and rear wall 25, a top 22 and bottom wall 26, two opposing side walls, 23 and 24, an inner horizontal dividing wall 28, and an inner vertical dividing wall 27 with a mounting hole 29 in the center, preferably, each wall with a planar surface on both sides. The assembly of the top 22 and its opposing bottom wall 26, the front 21 and its opposing rear wall 25, and two opposing side walls (23 and 24) forms the exterior of the housing 12. A heating compartment 15, located in the top of the housing 12, is formed by the inner horizontal dividing wall 28 and the inner vertical dividing wall 27 together with the top wall 22 and two opposing side walls (23 and 24). The heating compartment 15 is in the shape of a rectangular prism with the front side open as the front access opening 13. The housing walls of the heating device 2 can be made from various materials, including, for example, but not limited to, wood, stone, ceramic, tile, clay, heat resistant plastic and composite materials, metal, or combinations thereof. Preferably, the housing walls are made from a thermal insolating material or a material having a thermal insolating layer to keep the exterior surface cool enough to prevent burn injuries. The heating element 11 is securely mounted to the vertical dividing wall 27 by inserting the inner end of the heating element 11 through the center mounting hole 29 on the vertical dividing wall 27.

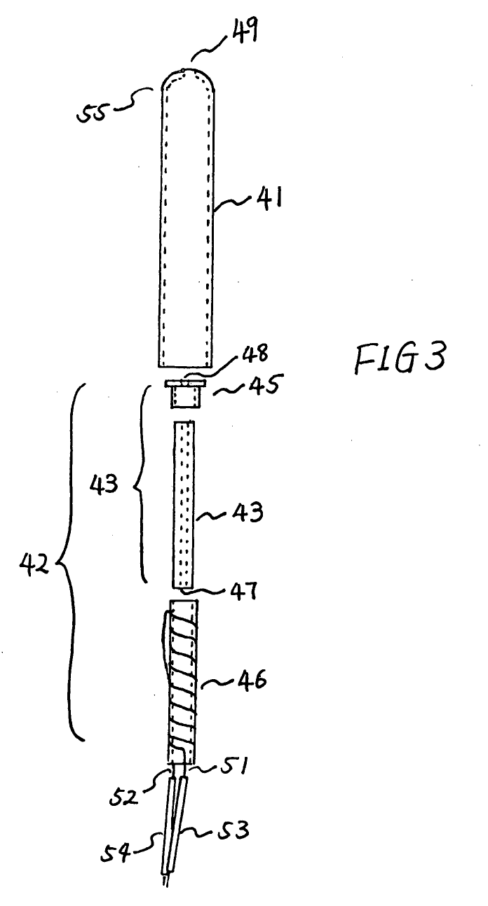

In certain embodiments, the heating element 11 includes a tubular protection shield 41 and an inner core assembly 42 (FIG. 3). The inner core assembly 42 is a heating coil which includes an inner metal core 43 having a cylindrical shank 44 connected to a cylindrical head 45. The inner metal core 43 has a passage 47 along the axial center and an annular opening 48 on the distal end of the cylindrical head 45 to allow air to flow through during inhalation. An insulated heating coil assembly 46 is then mounted over the shank 44 of the inner metal core 43. The heating coil assembly 46 also includes two wires, 51 and 52, for power supply, with one wire connected to the voltage controller of the switch 14 and the other directly to the power cord 16 located on the rear wall 25.

The tubular protection shield 41 has a tapered end with an aperture 49 for air to pass through during inhalation. Preferably, the protection shield 41 has an inner diameter slightly larger than that of the cylindrical head 45. The aperture 49 on the tapered end of the outer shield 41 has a smaller diameter than that of the cylindrical head 45 to prevent the inner core assembly sliding out the protection shield 41. The tubular protection shield 41 can be made from various thermal insulating and heat resistant materials, including, for example, but not limited to, stone, ceramic, tile, clay, heat resistant plastic and composite materials, or combinations thereof. The inner core assembly 42 is positioned into the outer shield 41 by sliding the core assembly 42 into the cylindrical bore of the shield 41 until the cylindrical head 45 abuts the tapered end of the shield 41, thus form a heating element 11.

The heating element 11 can be constructed from a variety of heating coils with different configurations and capacities. Some suitable examples of heating coils include soldering irons which are commercially available. The heating coil may have a power rating of from about 5 to about 300 watts, from about 5 to about 250 watts, from about 5 to about 200 watts, from about 5 to about 150 watts, from about 5 to about 100 watts, from about 5 to about 30, from about 5 to about 28 watts, from about 5 to about 26 watts, from about 5 to about 24, from about 5 to about 22 watts, from about 5 to about 20 watts, from about 30 to about 250 watts, from about 35 to about 250, from about 35 to about 200, from about 35 to about 150 watts, from about 35 to about 100 watts, from about 40 to about 250, from about 40 to about 200 watts, from about 40 to about 150 watts, from about 40 to about 100, from about 50 to about 250, from about 50 to about 200 watts, from about 50 to about 150 watts, or from about 50 to about 100. With improvements in heating coils or with different configurations of the heating device 2, the requirement for the power rating of the heating element 11 may have to be changed to produce appropriate amount of heat for the thermal vaporizer apparatus 1 of the present invention.

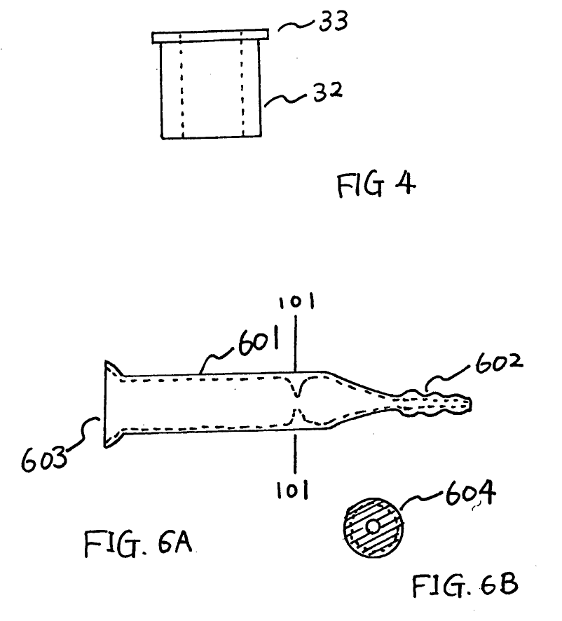

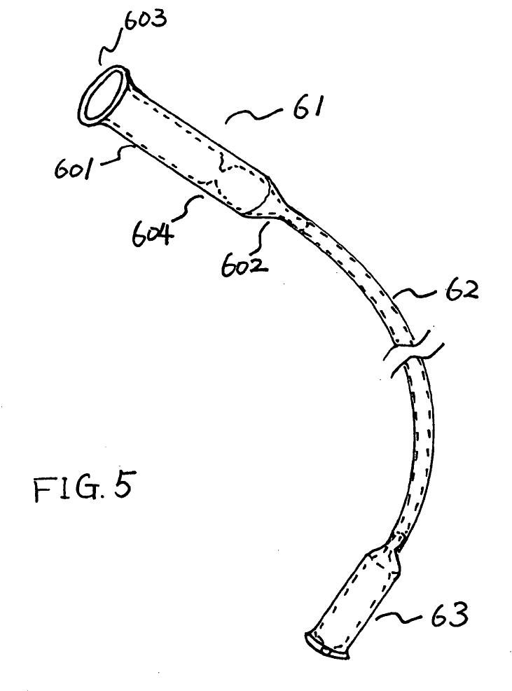

The heating element 11 is mounted securely to the heating device 2 through a retainer 31 which snuggly fits into the mounting hole 30 on the vertical dividing wall 27, preferably parallel to the top wall 22 and the horizontal dividing wall 28. The mounting hole 30 has a diameter larger than the outer diameter of the protection shield 41 of the heating element 11 but smaller the outer diameter of the retainer 31. Preferably, the retainer 31 has a cylindrical body 32 and a cylindrical head 33 (FIG. 4). The cylindrical head 33 has a diameter larger than the mounting hole 30 to prevent the retainer 31 slipping through the mounting hole 30 during installation. The retainer 31 also has a smaller inner diameter than the outer diameter of the protection shield 41 to provide frictional engagement between the retainer 31 and the protection shield 41. Preferably, the retainer 31 is made from an elastic and durable material, for example, such as rubber.

The inner core assembly 42 is securely held within the protection shield 41 with the help of a pair of stiffsleeves, 53 and 54, with appropriate lengths over the two exposed ends of the two electrical wires, 51 and 52, of the inner core assembly 42. The lengths of the sleeves, 53 and 54, are determined based on the length of the protection shield 41 and the position of the elongated hearting element 11 within the heating chamber 15 after installation. Preferably, the whole body of the heating element 11 is all disposed within the heating compartment 15 with the aperture 49 on the tapered end of the heating element 11 sufficiently proximate to the access opening for engagement with the inlet end 603 of vaporization chamber 61 of the handheld vapor delivery device 3.

In certain embodiments, the heating element 11 is mounted at an angle relative to the horizontal surface when the heating device 2 is place on a horizontal surface. The angle is defined by the longitudinal side of the heating element 11 and the horizontal surface, which can be the bottom wall 26 when it is parallel to the horizontal surface. The angle can be in a range of from about 2° to about 30°, 28°, 26°, 24°, 22°, 20°, 18°, 16°, or about 15°; from about 4° to about 30°, 28°, 26°, 24°, 22°, 20°, 18°, 16°, or about 15°; from about 6° to about 30°, 28°, 26°, 24°, 22°, 20°, 18°, 16°, or about 15°; from about 8° to about 30°, 28°, 26°, 24°, 22°, 20°, 18°, 16°, or about 15°; or from about 10° to about 30°, 28°, 26°, 24°, 22°, 20°, 18°, 16°, or about 15°. When the angle is appropriately selected, the heating element 11 can maximize the heat efficiency by preventing unnecessary heat loss from both the heating compartment 15 and the heating element 11 as well, thus potentially reducing the vaporization time.

In certain embodiments, the top wall 22, two opposing side walls (23 and 24), the rear wall 25 were assembled together along their edges. Preferably, the two opposing side walls (23 and 24), which are parallel to each other, are vertical to the top wall 22 and the rear wall 25. The nearly vertical dividing wall 27 was then inserted along two nearly vertical grooves, each on the interior side of each side wall. Preferably, the vertical dividing wall 27 is vertical to both the top wall 22 and the two opposing side walls (23 and 24). The horizontal dividing wall 28 is inserted along two horizontal grooves, each on the interior side of each side wall. Preferably, the horizontal dividing wall 28 is vertical to the two opposing side walls (23 and 24) but parallel to the top wall 22. The horizontal dividing wall 28 is long enough so that the bottom edge of the vertical dividing wall 27 securely sits on the top surface of the horizontal dividing wall 28 along its rear edge. As such, the heating compartment 15 with the shape of a rectangular prism is formed. The front wall 21 with a switch 14 was then mounted as such that the top edge is aligned with the top surface of the horizontal dividing wall 28. Preferably, the front wall 21 is vertical to the top wall 22 and two opposing side walls (23 and 24). After installation of the heating element 11 and electric circuitry 29, the flat bottom wall 26 was securely mounted to the bottom of the housing 12 along the rectangular recess edge formed by the bottom edges of the front wall 21, the rear wall 25, and the two opposing side walls (23 and 24). The bottom wall 26 also serves as a recess panel when maintenance is required.

In another embodiment, the heating device 2 further comprises a height adjustment control 17 (FIG. 9). The height adjustment control can be used to adjust the angle of the heating element 11 relative to the horizontal surface 18 to adapt to particular applications of the present inventions. The angle is similarly defined by the longitudinal side of the heating element 11 and the horizontal surface 18. Through suitable design of the housing 12, the angle can be changed in a range of from about −45° to about 45°, from about −30° to about 30°, from about −25° to about 25°, from about −20° to about 20°, from about −15° to about 15°, from about −10° to about 10°, or from about −5° to about 5°. The tapered end 55 of the heating element 11 points downward when the angle is negative and upwards when the angle is positive. If desired, the heating device 2 can be designed to have only positive values for the angle, such as, greater than about 45°, no greater than about 30°, no greater than about 25°, no greater than about 20°, no greater than about 15°, or no greater than about 10°.

### Handhold Delivery Device

The handhold delivery device 3 comprises a vaporization chamber 61, a vapor delivery hose 62, and an inhalation mouthpiece 63. The vapor delivery hose 62 is a flexible tubing, such as a clear vinyl or plastic tubing, with one end connected to the vaporization chamber 61 and the other end to the inhalation mouthpiece 63. Preferably, the vaporization chamber 61 and the mouthpiece 63 are made out of boro-silicate glass.

In one embodiment, the vaporization chamber 61 has a tubular body 601 with a tapered outlet end 602 for coupling with the vapor delivery hose 62 and an inlet end 603 that is dimensioned to mate with the tapered end 55 of the heating element 11 of the heating device 2. The inlet end 603 can be tapered or flared. With a flared inlet 603, the vaporization chamber 61 can be held in various angles with respect to the longitudinal body of the heating element 11 without significantly hindering the air flow from the heating element 11 to the vaporization chamber 61. This design feature is also very useful in preventing inadvertent spillage of the packed herbal plants from the vaporization chamber 61.

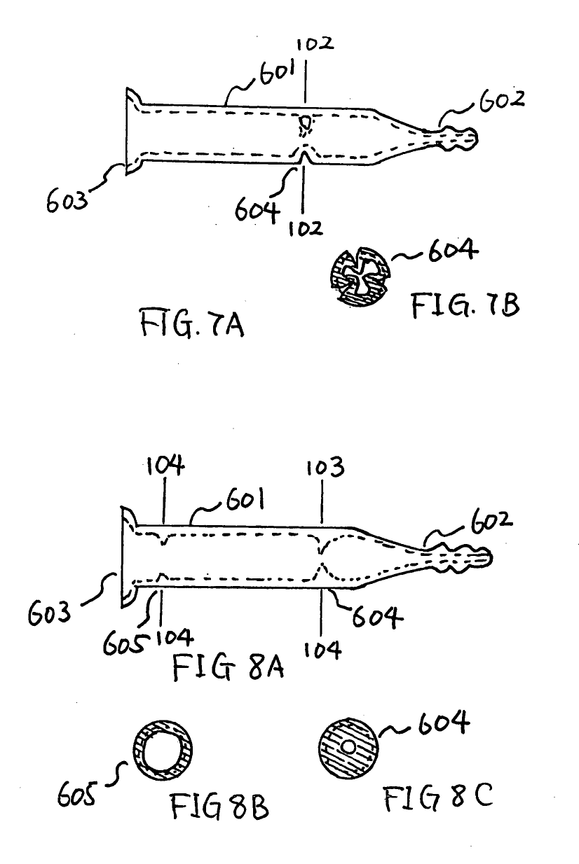

The vaporization chamber 61 also has a barrier 604 close to the taper end 602, to prevent the packed herbal materials or other particles in the vaporization chamber from being withdrawn into the vaporization delivery hose 62 and/or the inhalation mouthpiece 63. The barrier 604 can be a replaceable mesh screen or filter fitted into the cylindrical bore. One common problem with a mesh screen or filter is that the screen or filter gets clotted after a period of use and requires replacement. Another problem is that the screen requires a tedious daily cleaning, which is also especially difficult due to limited access. To circumvent these problems, the barrier 604 of the present invention does not have a screen or filter. The screen-less barrier 604 can be an integral section of the tubular body of the vaporization chamber 61 which has a reduced dimension to minimize passage of the packed herbs and other particles. The barrier 604 can be formed simply by reducing the inner diameter of a section of the vaporization chamber 61 (FIG. 6). The barrier 604 can also be a dimpled section of the vaporization chamber 61 (FIG. 7). The screen-less design of the barrier 604 in the present invention completely eliminates the clotting problem and significantly and simplifies the cleaning process. In some special cases, if needed, a single-use, disposable filtration material, such as cotton fibers or glass fibers, can also be used in the design of the screen-less barrier 604 to prevent fine particles from entering the vapor delivery hose 62 and/or the inhalation mouthpiece 63.

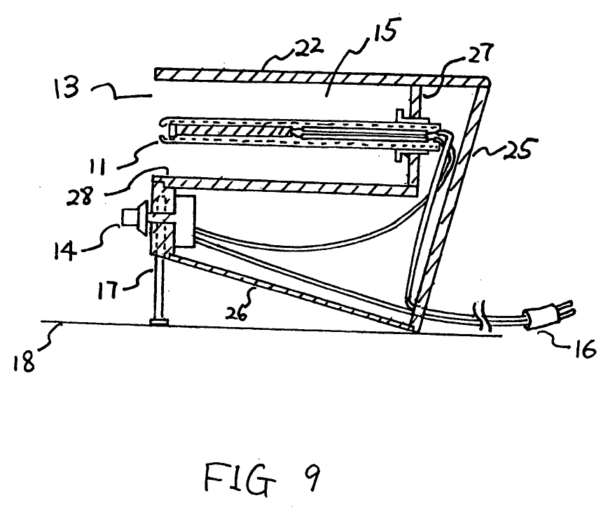

In another embodiment, the vaporization chamber 61 includes a second barrier 605 close to the inlet end. Preferably, the second barrier 605 is a section of the cylindrical body of the vaporization chamber 61 with a slightly reduced diameter to minimize the inadvertent spillage of the packed herbal plants from the vaporization chamber 61 (FIG. 8).

### Operation

A desired amount of herbal plants is packed into the vaporization chamber 61 through the inlet end 603. The vaporization chamber 61 is held in the position to mate with the tapered end of the heating element 11. The selection of voltage on the variable voltage controller is determined by factors such as the moisture content in the herbal plant, the volatility of the active ingredients for vaporization, and the heat output of the heat element. In one exemplary embodiment, the voltage controller is set to the maximum for an appropriate period of time, with the loaded vaporization chamber 61 engaged to the heating element 11, for the maximum heat output. The voltage controller is then turned to the half way so that the heat output is reduced to avoid overheating the packed herbal plants. This mode of operation significantly reduces the overall heating time and avoids causing overheat. It has been found that a satisfactory heating temperature is reached about 20 to 30 sec using a heating element from a conventional soldering gun with a rated output of about 30 watts. Once a satisfactory heating temperature is reached, the hot air is drawn into the vaporization chamber 61 from the heating element 11 through the tapered opening of the heating element 11 and the inlet end 603 of the vaporization chamber 61 and initiates the extracting and vaporizing process. Through controlled breathing, the user is able to control the speed of the air flow through the vaporization chamber 61, thus the speed of the extraction and vaporization.

There are enormous varieties of plants from which therapeutically active components can be extracted and vaporized using the thermal vaporizer apparatus 1 of the present invention for inhalation. For example, eucalyptus leaves can be used as a decongestant or cough suppression and tobacco leaves can be used to treat smoking addiction. It is important to understand that the therapeutically active compounds that are inhaled can be used to treat a variety of deficiencies and maladies, including those that do not involve pathos of the lungs.

One skilled in the art will understand that various modifications may be made to the above embodiments which are still within the scope and spirit of the invention described herein. For example, the vaporization chamber 63 can have a variety of shapes without fundamentally affecting the desired functions of the apparatus. Therefore, the above description should not be construed as limiting but merely as exemplifications of the preferred embodiments of the invention.

1. A smokeless thermal vaporizer apparatus for extracting and vaporzing the volatile essential ingredients from herbal plants, the apparatus comprising:
a beating device which comprises:
a housing formed by a plurality of housing walls, having a heating compartment with a front access opening;
a heating element mounted securely within the heating compartment;
a switch with a variable voltage control, which is mounted to the front wall of the housing to control the voltage of the electricity supplied to the heating element; and
an electric circuit for power supply.
